(19) United States
(12) Patent Application Publication
(10) Pub. No.: US 2004/0187893 A1
(43) Pub. Date: Sep. 30, 2004

(54) Scrubbing element with enzyme/hydrophilic

(76) Inventors: Walter L. Maguire JR., Guilford, CT (US); Shaun Sweeney, Hamburg, NJ (US)

Correspondence Address: ST. ONGE STEWARD JOHNSTON & REENS, LLC, 986 BEDFORD STREET, STAMFORD, CT, 06905-5619, US

(21) Appl. No.: 10/403,580

(22) Filed: Mar. 31, 2003

(51) Int. Cl. B08B 9/04

(52) U.S. Cl. 134/8; 134/22.11; 134/26; 15/21.1

(57) ABSTRACT

A cleaning element comprising a brush having plurality of bristles coated with a hydrophilic polymer. The cylindrical design allows the brush to be pushed through a surgical instrument or lumen. The brush allows for beneficial cleaning of surgical lumens. Also, a method for cleaning surgical instruments or lumen by pushing a brush through a surgical instrument is disclosed.

### FIELD OF THE INVENTION

The invention relates to cleaning elements and methods of using the elements for cleaning surgical devices.

### BACKGROUND OF THE INVENTION

The present invention is in the field of cleaning surgical instruments, such as flexible and nonflexible endoscopes, surgical lumens, and other medical paraphernalia used in healthcare facilities. This cleaning typically takes place as a preliminary step subsequent to use and soiling of the surgical instruments, and prior to their sterilization.

The physical designs of most models of endoscopes do not make possible the cleaning of every internal surface. It has been a common practice to merely soak used surgical instruments such as biopsy channels or lumens in a detergent bath and scrubbing with a small scrub brush prior to their being sterilized. Since delicate material is often used to make flexible endoscopes, scrub brushes longer than about 2.0 cm have a tendency to damage the lumen. Moreover, small scrub brushes cannot thoroughly scrub the internal surfaces of surgical lumens resulting in-contaminants remaining throughout surgical lumens. Pushing a scrub brush through a lumen is also problematic because it may damage the lumen wall.

There is concern about the transmission of diseases that commonly arise in healthcare facilities, and by viruses carried in tissues and blood, such as hepatitis B and HIV, which may be transmitted to other patients or personnel dealing with soiled surgical instruments such as most models of flexible endoscopes. This concern stems from the difficulty in cleaning surgical lumens by a method meticulous enough to scrub a soiled lumen, while at the same time being simple and straightforward enough to be utilized by personnel requiring a minimum amount of training, and using an apparatus which is both inexpensive and reliable. Moreover, it is problematic that surgical lumens and other paraphernalia may be grossly soiled, and therefore require vigorous cleaning throughout the entire length of the surgical lumen, both inside and out. Unfortunately, cleaning agents and brushes are not available which easily accomplish the vigorous cleaning of soiled surgical lumens prior to their microbiological decontamination. This problem is compounded by the fact that cleaning brushes are usually incompatible with flexible surgical lumens. Thus, heretofore, it has not been easy to clean the inside of deep surgical lumens using an elongated brush as described herein.

A number of different methods are known in the art for cleaning surgical instruments and other medical paraphernalia. For example, U.S. Pat. No. 4,867,797 herein incorporated by reference discloses a method for cleaning instruments used for analyzing protein-containing biological liquids which utilizes an enzyme rinse solution, but uses germicides therewith only in low concentrations and only to increase the stability of the enzyme composition by protecting it against microbial deterioration.

U.S. Pat. No. 6,224,544 relates to an endoscope, being insertable in the colon of a patient, in a self-propelled manner, by driving a plurality of endless belts mounted along the outside surface of a flexible section of an insertion tube thereof, and having a cleaning mechanism therein to be easily cleaned after use. The insertion tube, operation unit, and a lower portion of the driving unit casing are cleaned by immersion in a washing vessel filled with a cleaning solution.

U.S. Pat. No. 5,489,531 herein incorporated by reference relates to a two-stage method using the same container for both cleaning and microbiologically decontaminating grossly soiled surgical instruments. A presoak in an enzyme solution is followed by direct addition of a compatible disinfectant and a continued soak to decontaminate surgical instruments and other paraphernalia used in healthcare facilities.

Longitudinal brushes having a twisted wire core are known, such as, for example, mascara brushes used to apply mascara to a user's eyelashes. A typical mascara brush is made of a core formed from a single metallic wire folded in a generally u-shaped configuration to provide a pair of parallel wire segments. Bristles, usually made of strands of nylon, are disposed between a portion of a length of the wire segments. The wire segments are then twisted, or rotated, about each other to form a helical core (also known as a twisted wire core) that holds the filaments substantially at their midpoints so as to clamp them. In this way, a bristle portion or bristle head is formed with radially extending bristles secured in the twisted wire core in a helical or spiral manner. See, for example, U.S. Pat. No. 4,887,622, U.S. Pat. No. 4,733,425, and U.S. Pat. No. 6,481,445 all of which are herein incorporated by reference.

It is problematic that unclean instruments cannot be properly disinfected or sterilized. Moreover, the physical properties of endoscope tubing and the designs of some complex surgical instruments contribute to limiting the effectiveness and reliability of brush devices, detergent systems, and increases the probability that a lumens internal surfaces may remain contaminated following state-of-the-art cleaning. Accordingly, what is needed is a cleaning brush that provides a simple, cost efficient mechanism for cleaning surgical instruments by providing direct access to the internal surface, as well as cleaning brush kits and systems, which deliver cleaning solution directly to the deep channels of the lumens. cl SUMMARY OF THE INVENTION

Accordingly, it is an object of the present invention to provide a cleaning element, which is durable and flexible.

Accordingly, it is an object of the present invention to provide a cleaning element that provides a simple, cost efficient mechanism for cleaning surgical instruments by providing direct access to the internal surfaces of a lumen.

Accordingly, it is an object of the present invention to provide a cleaning element that increases the effectiveness and reliability of brush devices, and detergent systems.

Accordingly, it is an object of the present invention to provide a cleaning element that decreases the probability that a lumen's internal surface may remain contaminated following cleaning.

Accordingly, it is an object of the present invention to provide a cleaning element and methods of use thereof that promote sterilization through thorough cleaning.

Accordingly, it is an object of the present invention to provide a cleaning element and methods of use thereof that remove and dissolve, blood, fat, proteins, mucous and other organic contaminates from soiled surgical instruments and lumens.

Accordingly, it is an object of the present invention to provide a cleaning element and methods of use thereof that maintain contact and positive pressure between enzyme and lumen during soaking cycle.

Accordingly, it is an object of the present invention to provide a cleaning element and methods of use thereof comprising a full-length cylindrical bristle portion of brush, which effectively removes residual debris from interior of soiled lumens or surgical instruments.

Accordingly, it is an object of the present invention to provide a cleaning element and methods of use thereof comprising a bristle portion at least 0.15 m in length.

Accordingly, it is an object of the present invention to provide a cleaning element and methods of use thereof that do not damage instrument seals or rings.

Accordingly, it is an object of the present invention to provide a cleaning element and methods of use thereof that prevent corrosion of stainless steel and carbon steel surgical instruments.

Accordingly, it is an object of the present invention to provide a cleaning element and methods of use thereof that increase the effectiveness of the enzyme/surfactant cleaning properties.

Accordingly, it is an object of the present invention to provide a cleaning element and methods of use thereof that increases effectiveness of decontamination and sterilization of precleaned surgical instruments.

These and other objects of the present invention are achieved by provision of a cleaning element for lumen of surgical instruments comprising: a brush having a proximal end, a distal end, and a core, the core defining a longitudinal axis extending throughout a first length of the brush; a plurality of bristles fastened to the core to form a brush portion throughout a second length of the core; and a coating layer of hydrophilic polymer disposed upon the bristles.

In some embodiments the cleaning element is formed having a first length between about 0.15 m to about 4.0 m in length.

In some embodiments the cleaning element is formed having a second length between about 0.15 m to about 4.0 m in length.

In some embodiments the cleaning element is formed having a first length which is about equal to the second length.

In some embodiments the cleaning element is formed having a core having a diameter of from about 1 to about 5 mm, preferably about 1.5 mm to about 2.7 mm.

In some embodiments the cleaning element is formed having a core made of twisted wire.

In some embodiments the cleaning element is formed having core of twisted wire made of 0.010 inch diameter to about 0.020 inch diameter.

In some embodiments the cleaning element is formed having bristles made of material selected from the group consisting of polyester, polypropylene, cotton, woven fabrics, non-woven fabrics, nylon, polyamide derivatives, thermoplastic elastomers, and combinations thereof.

In some embodiments the cleaning element is formed having coating layer further comprises at least one reservoir.

In some embodiments the cleaning element is formed having a reservoirs or pores having an inner surface, wherein disposed upon the inner surface is at least one cleaning solution.

In some embodiments the cleaning element is formed having hydrophilic polymer as a coating layer disposed upon the bristles, such as hydrophilic urethane.

In some embodiments the cleaning element is formed having coating layer where the hydrophilic polymer is foam.

The objects of the present invention are further achieved by providing a method of cleaning a soiled lumen having a first end and a second end comprising:

(a) providing a brush having a proximal end, a distal end, and a core, the core defining a longitudinal axis extending throughout a first length of the brush; a plurality of bristles fastened to the core to form a brush portion throughout a second length of the core; and a coating layer of a hydrophilic polymer disposed upon the bristles;

(b) inserting the brush into the first end of the lumen to create an exposed brush segment;

(c) pushing the exposed brush segment away from the first end of the lumen so that the brush passes through the lumen.

In some embodiments the method of cleaning a soiled lumen further comprises the step of providing a brush comprising a plurality of bristles secured between twisted wire.

In some embodiments the method of cleaning a soiled lumen further comprises the step of providing a brush portion coated with a cleaning solution.

In some embodiments the method of cleaning a soiled lumen further comprises the step of selecting a brush having a brush portion with a diameter of from about 2.0 mm to about 20 mm.

In some embodiments the method of cleaning a soiled lumen further comprises the step of selecting a brush having a first diameter; comparing the first diameter with a lumen having a second diameter, wherein the first diameter and the second diameter are approximately equal.

In some embodiments the method of cleaning a soiled lumen further comprises the step of cleaning a soiled lumen with a brush having a coating layer made of hydrophilic polymer, wherein the hydrophilic polymer is hydrophilic urethane.

In some embodiments the method of cleaning a soiled lumen further comprises the step of rinsing the lumen with water.

In some embodiments the method of cleaning a soiled lumen further comprises the step rinsing the lumen with cleaning solution, or agent.

In some embodiments the method of cleaning a soiled lumen further comprises the step sterilizing the lumen.

In some embodiments the method of cleaning a soiled lumen further comprises the step of scrubbing the outside of the lumen.

In some embodiments the method of cleaning a soiled lumen further comprises the step of soaking the cleaning element with brush portion positioned within the biopsy channel.

The term “surgical instruments” as used herein means any of those instruments commonly used in a wide variety of surgical procedures, whether in a hospital operating room environment or in a doctor's office on an outpatient basis. Such instruments are, for the most part, made of surgical quality stainless steel, but they may be composed of other materials as well, e.g., aluminum and polypropylene and other polymer materials. In addition, the term “surgical instruments” includes other medical and surgical paraphernalia which might not typically be considered a surgical instrument, but which comes into contact with human tissue, especially blood, during a surgical or some other medical procedure, during the course of which that item of medical or surgical paraphernalia becomes grossly soiled and microbiologically contaminated. Examples of such medical and surgical paraphernalia are cardiovascular instruments, eye instruments, micro-surgical instruments, neurologic and orthopedic instruments, laparoscopes, flexible fiberoptic scopes, endoscopes, bronchoscopes, cystoscopes, colonoscopes, and respiratory therapy equipment.

The terms “grossly soiled” and “substantially soiled” as used herein mean the condition of being contaminated to a substantial extent by contact with human tissue, fluids, excretia, and so forth, as the result of contact therewith during some surgical or other medical procedure. Contamination by contact with human blood in substantial amounts is particularly referred to, and this includes microbiological contamination by viruses and bacteria contained in that blood. A surgical instrument that is “grossly soiled” is one that requires a step of cleaning prior to reuse. The step of cleaning removes or lifts the human tissue, fluids, excretia, etc. which have adhered to the surgical instrument, but provides little or no microbiological decontamination of the viruses, bacteria or other microorganisms present in that human tissue, fluids, excretia, etc., which have also adhered to the surgical instrument to be cleaned. For these, the further step of microbiological decontamination is needed.

The term “lumen” means the bore of a tube. The term also means tubing of or from a surgical instrument as defined above. In addition, the term includes ‘biopsy channel’ and other medical and surgical tubing which might not typically be considered a lumen, but which comes into contact with human tissue, especially blood, during a surgical or some other medical procedure, during the course of which that item of medical or surgical tubing becomes grossly soiled and/or microbiologically contaminated.

As used herein, the term “hydrophilic”, when used in connection with a solid, means capable of being readily wet by water. When used in connection with a liquid, the term “hydrophilic” means the liquid is miscible in water or aqueous solutions.

As used herein “enzyme-based cleaning compositions” refers to cleaning compositions designed to remove substantially all human tissue, fluids, excretia, etc. from grossly soiled metal and other surfaces, especially surgical instruments and other medical paraphernalia, in which the enzyme is selected from protease, lipase, amylase, carbohydrase or other enzymes or combinations of enzymes and surfactants known to break down blood, body tissue and excretia.

The term “germicidal detergent microbiological decontamination composition” refers to germicidal detergents especially designed to provide microbiological decontamination of all grossly soiled metal and other surfaces, especially surgical instruments and other medical paraphernalia, selected from phenolic compounds, quaternary amines, glutaraldehyde and other known disinfectants or combinations thereof.

As used herein the “urethane” is an ester of carbamic acid; also a monoester, monoamide of carbonic acid.

### BRIEF DESCRIPTION OF THE DRAWINGS

FIG. 1 is a side view of a brush in accordance with the present invention showing the bristle portion in representative form and further provides a side view of a lumen;

FIG. 2(a) is a side view of a brush in accordance with the present invention with a leader, showing the bristle portion in representative form;

FIG. 2(b) is a side view of a brush accordance with the present invention showing a leader portion in a form different than FIG. 2(a);

FIG. 3 is a side view of a brush with leader as shown in FIG. 2(a);

FIGS. 4(a)(b)(c)(d) are perspective views of steps in the process of cleaning a surgical instrument of one embodiment of the present invention;

FIGS. 5(a)(b)(c) are perspective views of steps in the process of cleaning a surgical instrument with an embodiment of the invention without a leader and different than the embodiment shown in FIG. 4.

FIG. 6 is a perspective view of a step in the process of making a brush of the type shown attached to a leader in FIG. 1;

FIGS. 7(a)(b)(c)(d) are side views of a brush in accordance with the present invention showing the bristle portion in representative form; and

FIG. 8 is a side view of a kit including a brush as shown in FIG. 1, 2(a), and 2(b) (without leader) showing the kit in representative form;

FIG. 9 is an enlarged view of a portion of FIG. 2(b), showing side view of an individual bristle;

FIG. 10 is an enlarged view of a single bristle covered by a coating layer of brush of FIG. 1.

### DETAILED DESCRIPTION OF THE DRAWINGS

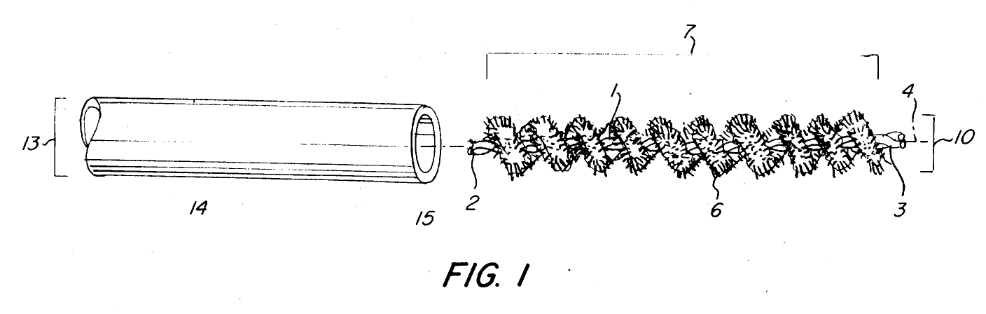

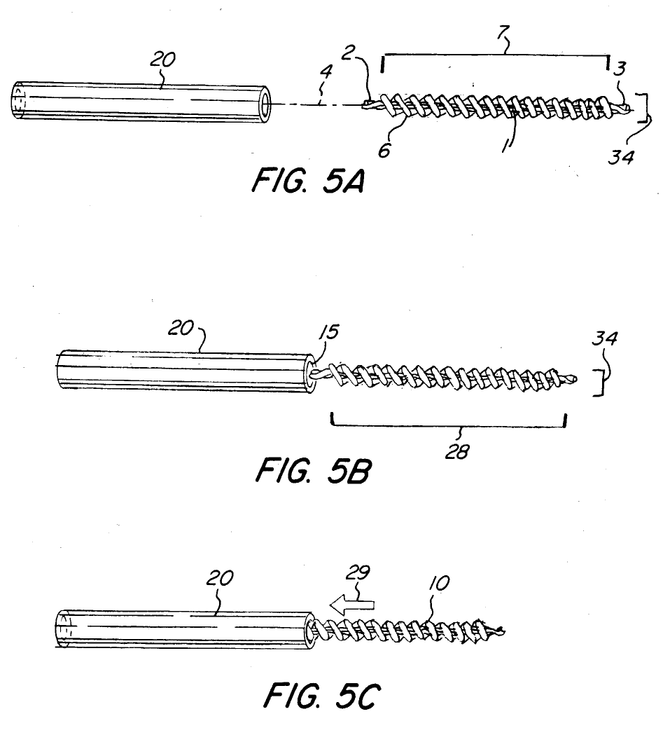

Referring now to FIG. 1, brush 10 is an elongated brushlike structure having a deformable core 1 of wire or other material. The brush 10 is intended for use in an instrument of the type having a tube 14 defining a lumen 13 with a first opening 15 to tube 14 dimensioned to receive brush 10. Brush 10 is comprised of a central core 1 or flexible wire having a first or proximal end 2, and a second or distal end 3 opposite the first end 2 in which bristle 6 of tufted fabric is twisted to form a bristle portion 7. A longitudinal axis 4 is defined along core 1 through first and second ends, 2 and 3, respectively. Bristle portion 7 extends along at least part of the length of the core 1 from first end 2 toward second end 3. Brush 10 is comprised of radially extending bristles 6 attached to core 1. Core 1 may be a twisted wire core made by first forming a pair of parallel wire segments 21 (FIG. 5) connected at one end 22 to form a “U”. Preferably, core 1 is interwoven with bristle 6 material to form a pipe cleaner type cleaning element.

Bristle 6 may be made by any known technique and from a variety of materials. For example, bristle 6 may be made from polyester, polypropylene, cotton, woven fabrics, non-woven fabrics, nylon or polyamide derivatives, thermoplastic elastomers, and combinations of these. Bristle 6 may be manufactured to a desired length, or may be cut to a desired length from a continuous filament. Bristle 6 or continuous filament may be selected from any one of a number of commercially available products that are made from a relatively soft thermoplastic elastomer material having various tear strengths, densities, and hardness. It is important that bristle 6 be flexible enough to be compressed when bristle portion 7 is squeezed between fingers of the user of any embodiment.

One preferred embodiment utilizes a less flexible core 1 by making brush 10 with a stiffer wire e.g. 0.02 inch diameter. Such an embodiment is desirable for cleaning stainless steel surgical instruments. It has been found that using a polyester yarn to form bristle 6 makes brush 10 more durable for this embodiment. Moreover, it has been found that a polyester yarn is a preferred substrate for applying a hydrophilic polymer such as hydrophilic urethane for this embodiment.

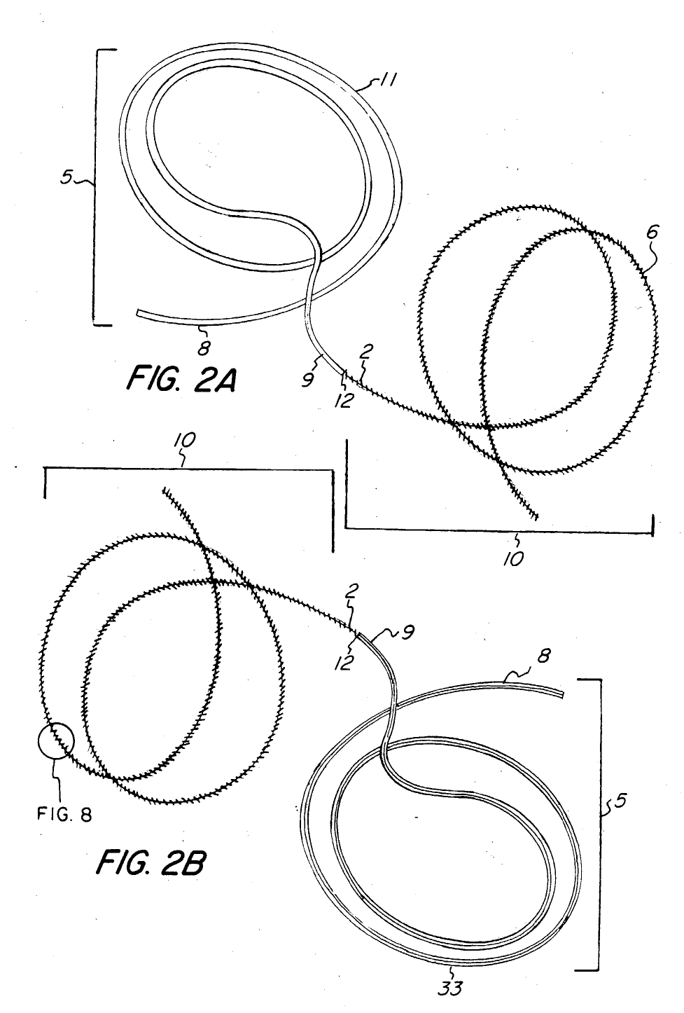

Referring to FIGS. 2(a)(b) & 3, another preferred embodiment utilizes a more flexible core 1 by assembling brush 10 with a flexible wire e.g. 0.014 inch diameter and/or 0.016 inch diameter. Such embodiments are desirable for cleaning flexible surgical instruments or lumens, and preferably utilize a polyester and/or polypropylene yarn to form bristle 6 on brush 10.

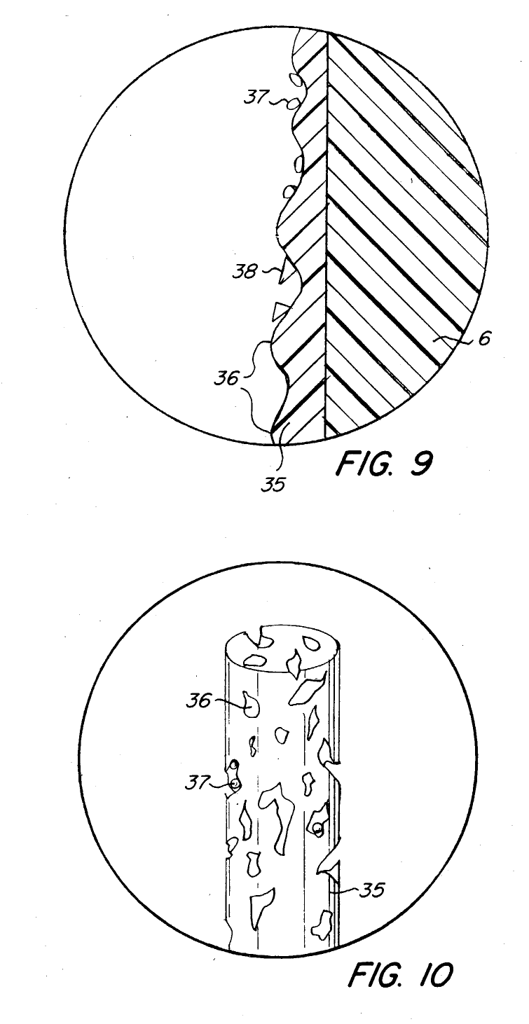

Referring now to FIGS. 9 & 10, one embodiment of the present invention provides a coating layer 35 on bristle 6 preferably comprising a hydrophilic polymer. In one embodiment, coating layer 35 preferably comprises a hydrophilic polymer such as hydrophilic urethane. Although not wishing to be bound by this disclosure, it is believed that providing a coating layer 35 on bristle 6 increases the surface area of bristle 6 and improves water absorption properties. Furthermore, coating layer 35 provides one or more pores or reservoirs 36. These pores 36 or reservoirs may hold cleaning solution 37, agents and mixtures thereof. Cleaning solution 37 does not wash out easily from the bristle 6 or bristle portion 7 because it is entrained throughout all the interstitial small pore spaces 36 of bristle 6. Such a design makes available the premanufacturing of cleaning elements of the present invention loaded with non-ionic detergents and a variety of multi-tier enzymes designed to target the most common bio-burden of flexible endoscopes, or lumens. Pore 36 may also hold disinfectant 38.

Materials suitable as cleaning agent 37 for the cleaning element of the present invention are manifold, provided that the materials are capable of promoting debris removal from grossly soiled and/or substantially soiled surgical instruments. Examples of such materials include detergents e.g. nonionic detergent. Suitable nonionic detergent active compounds can be broadly described as compounds produced by the condensation of alkylene oxide groups, which are hydrophilic in nature, with an organic hydrophobic compound which may be aliphatic or alkyl aromatic in nature. Such non-ionic detergents are readily known in the art and partially described in U.S. Pat. No. 6,492,321 herein incorporated by reference. It is also possible to include cationic, anionic, amphoteric, or zwitterionic detergent actives, or combinations of these, in the cleaning element according to the invention, however these are less preferable since they may break down the enzymes that may be present in the cleaning solution. Many suitable detergents are commercially available. It is believed that non-ionic detergents have a lower surface tension than water which helps to lift debris from surface of grossly or substantially soiled medical instruments. For example, nonionic detergent, and a wetting agent, will lift blood from the surface of a medical instrument, and break the blood apart in solution.

Additional materials suitable as cleaning agents include enzyme-based cleaning compositions. These enzyme-based cleaning compositions are well known in the art and are commercially available. The enzyme-based cleaning composition may have a number of additional ingredients which help promote its effectiveness and use, e.g., other cleaning agents such as sodium tetraborate, emulsifiers such as triethanolamine, solvent thickeners such as propylene glycol, acidifiers such as citric acid, buffering agents, preservatives, and so forth. Such excipients would be well known to one of ordinary skill in this art. One example of an enzyme-based cleaning composition suitable for use with the present invention is Enzymatic Cleaner sold by Enzyme Solutions Inc. of Hickory N.C., U.S.A. However any enzymatic cleaner, which promotes the removal of protein, blood, mucus, feces, urine, albumin, etc., from medical apparatus and instruments would be suitable.

Another embodiment of the present invention may further provide a disinfectant 38 or germicidal detergent microbiological decontamination composition applied to coating layer 35, or polymer layer. As shown in FIG. 9 disinfectant is preferably entrained throughout interstitial small pores 36 or reservoir spaces on coating layer of bristle.6. The disinfectant, i.e., the germicidal detergent decontaminating composition may have a number of additional ingredients which help promote its effectiveness and use, e.g., strong acids and bases such as phosphoric acid and caustic soda, emulsifiers and surfactants such as α-olefin sulfonate, and various fragrances which help to mask the odor of the phenolic compounds.

Referring now FIGS. 2(a)(b) & 3, brush 10 is shown attached to lead or leader 5. Leader 5 may be made by any known techniques and from a variety of materials. For example, leader 5 may be made from polyvinyl chloride, polystyrene, polyethylene, polyethylene terephthalate, rubber, natural rubber latex, acetal, butyrate, cast acrylic, ECTFE, extruded acrylic, polycarbonate, polyethylene, polypropylene, polysulfone, PVDF, nylon, polyurethane, thermoplastic (styrene-, propylene- and urethane-based) elastomers, and even the high-performance specialty resins such as silicone and fluoropolymers, as well as combinations of these materials. Leader 5 may be made from any suitable resin, plastic, or thermoplastic elastomer, and combinations thereof. Preferably leader 5 is made out of flexible polyvinyl chloride (PVC). Leader 5 may be made out of wire, or twisted wire segments. Leader 5 may be made out of a polymer thread similar to fishing wire. Leader 5 is typically flexible tubing precut to a desired length. Leader 5 has a proximal end 8 or first end, and distal end 9 or second end. The body portion 11 extends from first end 8 toward second end 9. Body portion 11 may be hollow. Leader 5 is typically between 0.2 m to 4 m in length from first end 8 to second end 9. Preferably leader 5 is long enough to be passed all the way through a lumen of known length. For example, if a lumen (not shown) for a colonoscope is 2 m in length, leader 5 would preferably be longer than 2 m in length, so that a user can pass first end 8 all of the way through the inside of the lumen so that the first end 8 extends out of the lumen opposite the receiving end of the lumen, while second end 9 remains below the receiving end of the lumen. Leader 5 may be substantially equal in length to brush 10 and brush portion 7. For example, if brush 10 is 2.0 m in length, leader 5 may be about 2.3 m in length. Leader 5 is of a predetermined diameter. The diameter of leader 5 is smaller than the diameter of the lumen so that leader 5 can pass through the inside of the lumen in which it has been inserted. Leader 5 has a diameter of from about 1.5 mm to about 6.0 mm, preferably about 1.5 mm to about 2.7 mm. Preferably leader 5 is about equal in length to brush 10 and brush portion 7. Leader 5 may be a hallow tube. Leader 5 is preferably cylindrical in shape.

It is important that leader 5 be made of material stiff enough to pass it through a soiled lumen. The desired stiffness may be achieved by utilizing a stiff polymer to form body portion 11, e.g. PVC, or Nylon. Support wire 33 may also be inserted within body portion 11 to increase rigidity of leader 5. Referring to FIG. 2(b), wire 33 is shown within leader 5. Wire 33 is of a type that is well known in the art, e.g., a conventional soft steel or iron wire, the dimensions and specifications of which are also well known. Wire 33 may be a 0.014 to 0.016 inch diameter wire, within the hollow body portion 11, however, any diameter wire may be utilized so long as it is able to flexibly pass through soft lumen tubing without damaging the lumen walls. Wire 33 preferably is of preselected length, however, preferably about equal in length to leader 5. Most preferably it is slightly shorter than leader 5 (within about 1 cm) so that it can lie within body portion 11, and proffer increased rigidity throughout the length of leader 5. Wire 33 is preferably a stainless steel braided wire with a nylon jacket or coating. Leader 5 may be entirely made up of wire 33 and attached directly to the core of brush portion as shown in FIG. 10. In such cases, leader 5 is preferably coated with a polymer layer or jacket preferably made of Nylon.

FIGS. 2(a)(b) & 3 show brush 10 attached to leader 5. Typically second end 9 of leader 5 is attached to first end 2 of core 1 to form joint 12. Joint 12 may be made by any known techniques using a variety of materials. For example, second end 9 may be tied onto the first end 2 of core 1 using a tie, string, or wrap (not shown). Alternatively, first end 2 of core 1 may be wrapped around second end 9 so that the two pieces are tied together. Moreover, first end 2 of core 1 and second end 9 may be bonded by utilizing a heat shrink wrap (not shown). Preferably the two ends are jointed using a bonding resin or adhesive cement. For example, Super Glue® or Krazy Glue® (brand adhesives may be utilized to form a bond. This type of bond is preferable since it allows the tips of both first end 2 and second end 9 to be bond directly together, avoiding a bulge that may form when other bonding methods are employed.

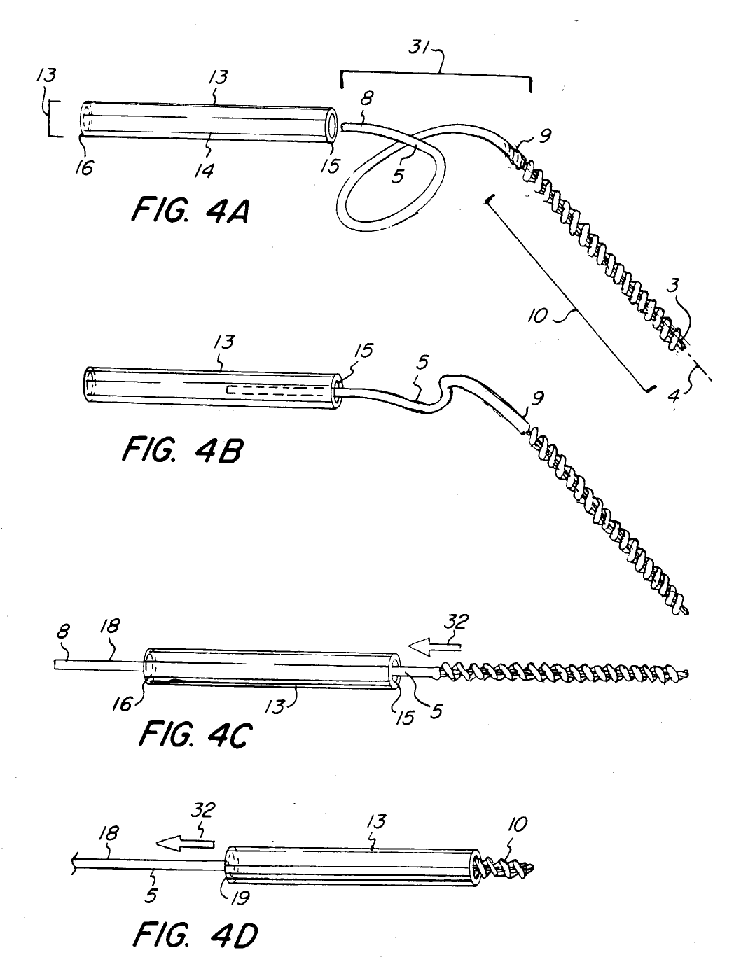

FIGS. 4(a)(b)(c)(d) show a method of using cleaning elements of the present invention. FIG. 4a shows cleaning element 31 with leader 5 next to a substantially soiled or grossly soiled lumen 13. Although not shown in the present drawings, lumen 13 is soiled and debris may randomly coat the inner walls of tube 14. In order to clean lumen 13 one must provide a cleaning element 31 of the present invention having brush 10, first end 2, second end 3, and a core 1, core 1 defining a longitudinal axis 4 extending throughout the length of brush 10; and leader 5 having first end 8, and second end 9. Second end 9 of leader 5 is connected to first end 2 of brush 10. FIG. 4b shows the step of inserting leader 5 into first end 15 of lumen 13. FIG. 4c shows the step of passing first end 8 of leader 5 through lumen 13 to create exposed leader portion 18, after distal lumen opening 16. Arrow 32 shows the direction of the movement of leader 5 through lumen 13. Even after leader 5 is passed through lumen 13, leader 5 is still visible below first end 15 of lumen 13. FIG. 4d shows the step of pulling exposed leader portion 18 away from second end 19 so brush 10 passes through lumen 13. Arrow 32 shows the direction of the movement of brush 10 through lumen 13, and confirms that the brush is being pulled through lumen 13. FIGS. 4(a)(b)(c)(d) show a method of using cleaning elements of the present invention. FIG. 4a shows cleaning element 31 with leader 5 next to a substantially soiled or grossly soiled lumen 13. Although not shown in the present drawings, lumen 13 is soiled and debris may randomly coat the inner walls of tube 14. In order to clean lumen 13 one must provide a cleaning element 31 of the present invention having brush 10, first end 2, second end 3, and a core 1, core 1 defining a longitudinal axis 4 extending throughout the length of brush 10; and leader 5 having first end 8, and second end 9. Second end 9 of leader 5 is connected to first end 2 of brush 10. FIG. 4b shows the step of inserting leader 5 into first end 15 of lumen 13. FIG. 4c shows the step of passing first end 8 of leader 5 through lumen 13 to create exposed leader portion 18, after distal lumen opening 16. Arrow 32 shows the direction of the movement of leader 5 through lumen 13. Even after leader 5 is passed through lumen 13, leader 5 is still visible below first end 15 of lumen 13. FIG. 4d shows the step of pulling exposed leader portion 18 away from second end 19 so brush 10 passes through lumen 13. Arrow 32 shows the direction of the movement of brush 10 through lumen 13, and confirms that the brush is being pulled through lumen 13. It has been surprising found that pulling the cleaning element of the present invention increases debris removal from soiled surgical instruments and lumen.

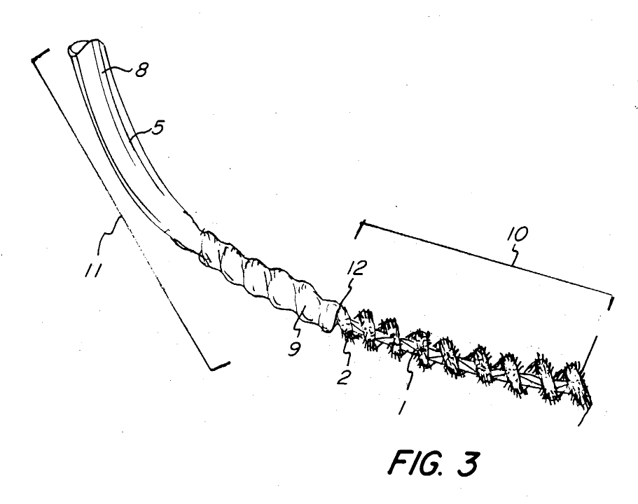

The method of use is not limited in any way to the aforementioned method. Other embodiments of the present invention may include the step of pushing a cleaning element 34 through a lumen 20 without the use of leader 5, as shown in FIGS. 1, 2, and 3. Referring now to FIGS. 5(a)(b)(c) show a method of using cleaning elements 34 of the present invention to clean soiled lumen 20 without a leader. FIG. 5(a) shows lumen 20 and brush 10 provided having first end 2, second end 3, and core 1, the core defining a longitudinal axis 4 extending throughout the length of the brush 10; a plurality of bristles 6 fastened to the core 1 form a brush portion 7 throughout the length of the core 1. FIGS. 9 & 10 show coating layer 35 of a hydrophilic polymer is disposed upon bristle 6. Coating layer 35 may be made of hydrophilic urethane and contain reservoirs or pores 36 on and/or within coating layer 35. Referring back, FIG. 5(b) illustrates the step of inserting brush 10 into the first end 15 of the lumen 20 to create exposed brush segment 28. FIG. 5(c) shows the step of pushing exposed brush segment 28 away from the first end 15 of lumen 20 so that brush 10 passes through lumen 20 in the direction of arrow 29. It has been surprising found that by pushing cleaning elements of the present invention at least 0.1 5 m in length into a soiled lumen or surgical instrument surprisingly promotes debris removal by supplementing the presence of cleaning solution within lumen, and/or maintaining contact and positive pressure between enzyme and lumen during soaking cycle. Such embodiments are especially suited for cleaning stainless steel surgical instruments, and preferably are comprised of a stiffer core material.

Other embodiments include the step of adding cleaning solution described above into lumen, as well as soaking the inserted cleaning element and lumen for two to five minutes in a bath to ensure that the brush 10 maintains positive contact with the interior walls of lumen 20 throughout the length of the channel. The soaking step further includes providing non-ionic detergents and a variety of enzymes designed to target the most common bio-burden of endoscopes and surgical lumens. As a dry, pre-dosed product, other embodiments of the claimed invention include storing the cleaning elements near the point of use and utilizing the cleaning element immediately after a procedure is performed with a surgical instrument. Other methods of use embodiments include the steps of wetting a soiled surgical instrument with tap water and inserting cleaning element into lumen. After soaking, the cleaning element is used to thoroughly brush the channel and remove debris such as protein, blood, mucus, feces, urine, albumin, etc., from all medical apparatus and surgical instruments.

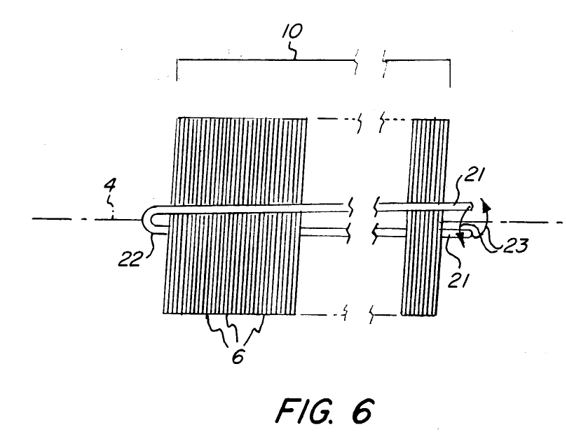

Referring back to the drawings, FIG. 6 shows a method for making brush 10 in accordance with the present invention. Core 1 (not shown) is a twisted wire core typically made by first forming a pair of parallel wire segments 21 connected at one end 22 to form a “U”. The wire is of a type that is well known in the art, e.g., a conventional soft steel or iron wire, the dimensions and specifications of which are also well known. A plurality of bristles 6 of a selected length and material (such as polyester or polypropylene) are placed between the pair of wire segments 21. The wire segments 21 are then twisted about the longitudinal axis 4 (see arrows 23 in FIG. 6) to secure in clamping engagement each of the bristles 6 at approximately a midpoint of bristle 6. In this way, opposite ends of each bristle 6 extend radially from the twisted wire core. After the bristles 6 are secured, the brush head may be trimmed by any suitable means, e.g., grinding, laser cutting, etc., to have any desired shape, e.g., cylindrical, tapered, conic, bi-conic, etc. Preferably the shape is cylindrical to ensure that greatest area of contact between brush 10, and the lumen 13 (of FIG. 1.). Most preferably, the brush portion may be made similar to how a pipe cleaner (e.g. for cleaning tobacco pipe) is made. Here the cleaning element is made in a continuous process using yarn instead of cut bristles, and the wire is not turned back on itself. Rather wire is interwoven with the yarn. This may be done using 50 to 2000 linear feet of yarn at one time.

Referring again to FIGS. 9 and 10, brush 10 of the present invention is preferably modified by coating bristle 6 with a coating layer 35, such as a hyprophillic polymer, e.g. hydrophillic urethane. Such embodiments may be formed by providing liquid coating solution such as hydrophillic polymer, prepared by any conventional technique. Moreover, a tub is provided long enough to hold a substantially straight brush. Tub may be between 0.1 m to 6 m in length. Liquid hydrophillic polymer solution is added to the tub in an amount adequate to form a bath and pan coat a brush. Brush is added to bath and allowed to sit for 30 seconds. It is also possible to simply run a spool of brush material through a bath using a manufacturing system, or alternatively use a conventional spray apparatus to apply the hydrophilic polymer to the brush. When the coating process is completed, the brush is cured. After the coating cures, a detergent rinse may be added to clean the coating layer. Brush is dried at an elevated temperature. Preferably the brush is dried at between 27 and 38° C. Most preferably brush is dried at 32.2° C. Coated brush is next cooled by supplying dry air of about room temperature for about 10 to 20 minutes. Brush coated with a hydrophilic polymer may then be further coated with cleaning solution, such as those described above, including non-ionic detergent, disinfectant, or an enzyme-based cleaning composition. Disinfectant may further be added to the coating layer. Other preferred embodiments using a leader require the step of fixing a leader of predetermined length and diameter to a brush of predetermined length and diameter. Preferably a leader is attached to brush by placing a 1 ml. of bonding resin such as Krazy Glue® to the first end of the brush, and fixing the second end of the leader to the first end of the brush.

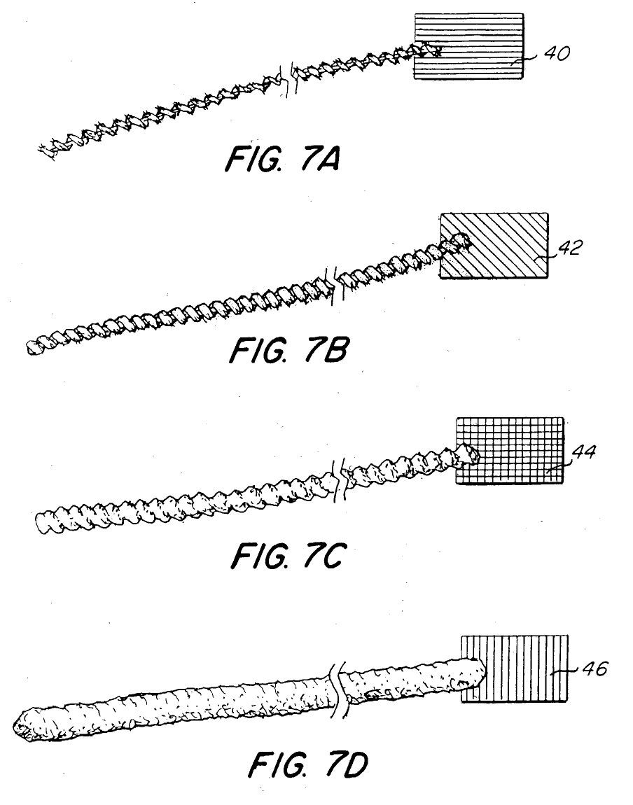

Other embodiments of the present invention include color-coded bristle portions of brush to aid the user in selecting the desired diameter brush portion. It is well known that lumens have a variety of known diameters. Brushes of the present invention are preferably pre-sized before use so that the brush portion diameter is about equal to the diameter of the lumen. The diameter of the brush portion is preferably slightly larger than the diameter of the lumen. Compressible bristles allow brush to easily fit into lumen. Color-coding brush portions aids non-technical persons in sizing the appropriate brush by simply matching a known brush color with a lumen of known dimensions. Referring back to the drawings FIG. 7(a) shows a blue (40) brush having a bristle portion of a first predetermined diameter. Preferably the first predetermined diameter is about 1.5 mm. FIG. 7(b) shows a green (42) brush having a bristle portion of a second predetermined diameter. Preferably the second predetermined diameter is about 2.5 mm. FIG. 7(c) shows a yellow (44) brush having a bristle portion of a third predetermined diameter. Preferably the third predetermined diameter is about 4.0 mm. FIG. 7(d) shows a red (46) brush having a bristle portion of a fourth predetermined diameter. Preferably the fourth predetermined diameter is about 6.0 mm. It should be understood that any color may be associated with any known diameter size and that FIG. 7(a-d) is illustrative and not limiting and that obvious modifications may be made by those skilled in the art without departing from the spirit of the invention.

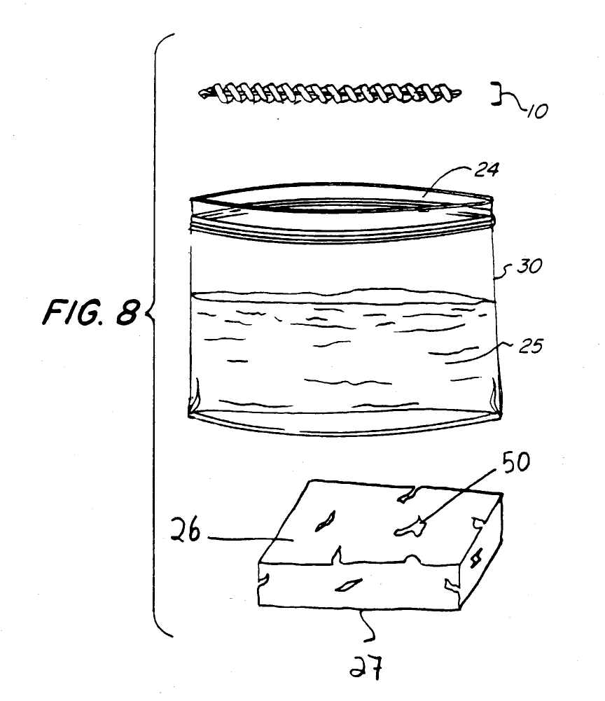

FIG. 8 shows a kit embodiment of the present invention. A kit for cleaning a soiled lumen having brush 10 of the present invention; a pouch 30 having an inner space 24 for holding liquid cleaning solution 25; a liquid cleaning solution 25; and/or a hand pad 26 having an outer surface 27. Liquid cleaning solution 25 may be a water-based buffer containing anti-microbial agents, and/or a water-based buffer containing detergents suitable for cleaning soiled surgical instruments, and/or a water-based enzyme cocktail. Inner space 24 is of a predetermined volume capable of holding between 10 to 500 ml of liquid cleaning solution, preferably about 75 ml. Hand pad 26 may be shaped to accommodate a lumen within the hand pad. Hand pad may have pores (50). Hand pad 27 may be made out of any sponge, or foam material suitable for wiping objects, such as natural sponge. Hand pad 27 may be flat. Preferably hand pad 27 is 3.5″×5.5″×⅜″ to ⅝″. Hand pad pad 27 may be die cut so it conforms to the scope. Had pad 27 facilitates cleaning of the outside of surgical instrument or lumen.

Cleaning elements and kits of the present invention provide the following benefits:

Removing and dissolving, blood, fat, proteins, mucous and other organic contaminates from soiled surgical instruments and lumens.

Maintaining contact and positive pressure between enzyme and lumen during soaking cycle.

Full-length cylindrical bristle portion of brush effectively removes residual debris from interior of soiled lumens or surgical instruments.

Decrease time required to clean surgical instruments.

Does not damage instrument seals or rings.

Prevents corrosion of stainless steel and carbon steel surgical instruments.

Increases the effectiveness of the enzyme/surfactant cleaning properties.

Increases effectiveness of decontamination and sterilization of precleaned surgical instruments.

It should be understood that the invention has been described for use with cleaning elements for the sake of convenience only and is not intended to be limiting. Other articles may be made in a similar manner after reading and understanding this disclosure.

It should be understood that the foregoing is illustrative and not limiting and that obvious modifications may be made by those skilled in the art without departing from the spirit of the invention. Accordingly, reference should be made primarily to the accompanying claims, rather than the foregoing specification, to determine the scope of the invention.

What is claimed is:

1. A cleaning element for lumen in surgical instruments comprising:
(a) a brush having a proximal end, a distal end, and a core, the core defining a longitudinal axis extending throughout a first length of said brush;
(b) a plurality of bristles fastened to the core to form a brush portion throughout a second length of the core; and
(c) a coating layer of hydrophilic polymer disposed upon said bristles.

2. The cleaning element of claim 1 wherein the first length is between about 0.15 m to about 4.0 m in length.

3. The cleaning element of claim 1 wherein the second length is between about 0.15 m to about 4.0 m in length.

4. The cleaning element of claim 1 wherein the first length is about equal to the second length.

5. The cleaning element of claim 1 wherein the core has a diameter of from about 0.5 mm to about 6.0 mm.

6. The cleaning element of claim 1 wherein the core is made of twisted wire.

7. The cleaning elements of claim 6 wherein the twisted wire is are made of from about 0.010 to about 0.020 inch diameter wire.

8. The cleaning element of claim 1 wherein the bristles are made of material selected from the group consisting of polyester, polypropylene, cotton, woven fabrics, non-woven fabrics, nylon, polyamide derivatives, thermoplastic elastomers, and combinations thereof.

9. The cleaning element of claim 1 wherein the coating layer further comprises at least one reservoir.

10. The cleaning element of claim 9 wherein the reservoir has an inner surface, wherein disposed upon the inner surface is at least one cleaning solution.

11. The cleaning element of claim 1 wherein the hydrophilic polymer is a hydrophilic urethane.

12. The cleaning element of claim 1 wherein the hydrophilic polymer is foam.

13. A method of cleaning a soiled lumen having a first end and a second end comprising:
(a) providing a brush having a proximal end, a distal end, and a core, the core defining a longitudinal axis extending throughout a first length of said brush; a plurality of bristles fastened to the core to form a brush portion throughout a second length of the core; and a coating layer of a hydrophilic polymer disposed upon said bristles;
(b) inserting the brush into the first end of the lumen to create an exposed brush segment;
(c) pushing the exposed brush segment away from the first end of the lumen so that the brush passes through the lumen.

14. The method of claim 13 wherein the step of providing a brush further comprises providing a brush further comprising a plurality of bristles secured between twisted wire.

15. The method of claim 13 wherein the step of providing a brush portion further comprises providing a brush portion coated with a cleaning solution.

16. The method of claim 13 wherein the step of providing a brush further comprises selecting a brush having a brush portion with a diameter of from about 2.0 mm to about 20 mm.

17. The method of claim 13 wherein the step of providing a brush further comprises selecting a brush having a first diameter; comparing said first diameter with a lumen having a second diameter, wherein said first diameter and said second diameter are approximately equal.

18. The method of claim 13 wherein said hydrophilic polymer is hydrophilic urethane.

19. The method of claim 13 further comprising rinsing the lumen with water.

20. The method of claim 13 further comprising rinsing the lumen with cleaning solution.

20. The method of claim 13 further comprising sterilizing the lumen.

21. The method of claim 13 further comprising scrubbing the outside of the lumen.

22. The method of claim 13 further comprising soaking the brush positioned within the biopsy channel.

23. A kit for cleaning a surgical instrument comprising:
(a) the cleaning element of claim 1;
(b) a pouch having an inner space for holding liquid solution; and
(c) a liquid cleaning solution.

24. The kit of claim 23 wherein said liquid cleaning solution is a water-based buffer.

25. The kit of claim 23 wherein said liquid cleaning solution is a water-based buffer containing detergent suitable for cleaning soiled surgical instruments.

26. The kit of claim 23 wherein said liquid cleaning solution is a water-based enzyme cocktail.

27. The kit of claim 23 wherein the inner space is of a predetermined volume capable of holding between 10 to 500 ml of liquid cleaning solution, preferably about 75 ml.

28. The kit of claim 23 further comprising a hand pad having an outer surface.
